(19) United States
(12) Patent Application Publication
(10) Pub. No.: US 2020/0121699 A1
(43) Pub. Date: Apr. 23, 2020

(54) COMBINATION THERAPIES COMPRISING IMMUNO-ONCOLOGY AGENTS AND BELINOSTAT

(71) Applicant: ONXEO, Paris (FR)

(72) Inventors: Graham DIXON, Saint Nom la Breteche (FR); Véronique TROCHON-JOSEPH, Vitry sur Seine (FR)

(21) Appl. No.: 16/078,706

(22) PCT Filed: Feb. 22, 2017
(86) PCT No.: PCT/EP2017/054081

§ 371 Date: Aug. 22, 2018

Related U.S. Application Data
(60) Provisional application No. 62/409,708, filed on Oct. 18, 2016.
(60) Provisional application No. 62/298,445, filed on Feb. 22, 2016.

(51) Int. Cl. A61K 31/63; A61P 35/00

(52) U.S. Cl. CPC A61K 31/63; C07K 16/2818; A61P 35/00

(57) ABSTRACT

The present disclosure pertains to combinations comprising immuno-oncology agents with belinostat or a pharmaceutically acceptable salt thereof, for use in treating various forms of cancer and in increasing anti-tumor activity and anti-tumor immune response in a patient in need thereof.

### BACKGROUND

The immune system is a collection of organs, cells, and special molecules that help protect the human body from infections and diseases, such as cancer. Immunotherapies refer to treatment methods that employ the immune system to fight diseases, and immuno-oncology therapies in particular employ the immune system to fight cancer, often by activating the immune system so that it is able to recognize cancer cells from normal cells so that the former may be targeted for destruction. Already approved by the U.S. Food and Drug Administration for advanced melanoma and non-small cell lung cancer, immune checkpoint inhibitors also appear to have significant antitumor activity in multiple other tumor types. An exciting component of immunotherapy is the durability of antitumor responses observed, with some patients achieving disease control for many years. Nevertheless, not all patients benefit, and efforts should thus now focus on improving the efficacy of immunotherapy through the use of combination approaches.

There remains a substantial unmet need to provide for new less-toxic methods and therapeutics that have better therapeutic efficacy, longer clinical benefit, and improved safety profiles, particularly for those patients with cancers that are resistant to existing therapeutics. Considering the benefits from immuno-oncology, the ability to combine immuno-oncology agents with other anti-cancer agents, particularly in synergistic fashion, would provide useful and improved combination therapies for treating various cancers in a large number of patients.

### SUMMARY

The present disclosure is directed to pharmaceutical combinations comprising one or more immuno-oncology agents, such as immunomodulatory compounds, with one or more anti-cancer agents, especially belinostat. For instance, in certain embodiments, the present pharmaceutical combinations comprise an immunomodulatory compound that is an antibody, as described herein, with belinostat or a pharmaceutically acceptable salt thereof.

In a first aspect, the invention relates to a pharmaceutical combination comprising at least one immunomodulatory compound and belinostat or a pharmaceutically acceptable salt thereof.

In a second aspect, the invention relates to a pharmaceutical combination as described above for use in a method for treating cancer in a patient in need thereof.

In a third aspect, the invention relates to a method for treating cancer in a patient in need thereof, comprising administering to the patient a therapeutically effective amount of the pharmaceutical combination as described above.

In a fourth aspect, the invention relates to a method for treating HCC in a patient in need thereof, comprising administering to said patient a therapeutically effective amount of HDACi inhibitor and a therapeutically effective amount of an anti-CTLA-4 antibody.

In a fifth aspect, the invention relates to a method for increasing anti-tumor activity in a patient suffering from cancer comprising administering to the patient a therapeutically effective amount of belinostat and a therapeutically effective amount of an immunomodulatory compound.

### BRIEF DESCRIPTION OF THE FIGURES

FIG. 1 provides the results for the combination of belinostat and anti-CTLA-4 antibody in a hepatocellular carcinoma (HCC) mouse model (Tumor growth in grouped mice treated with Belinostat (Beleodaq), anti-CTLA-4 or the combination).

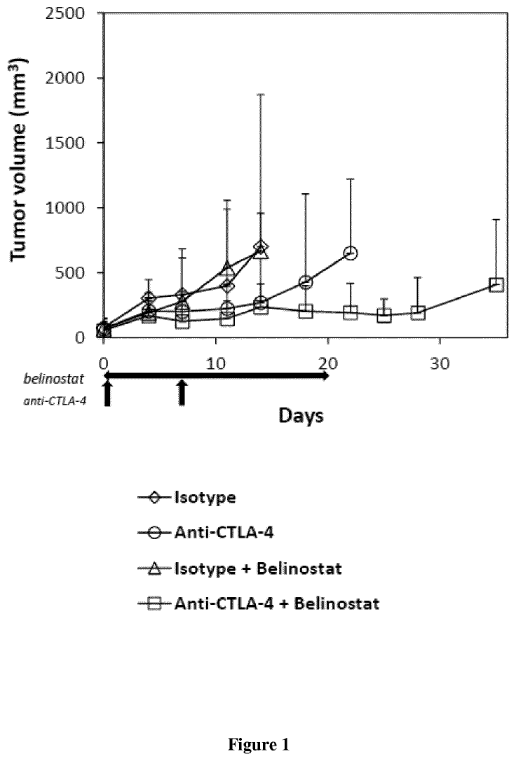

FIG. 2. shows the tumor growth in individual mice treated with Belinostat (Beleodaq), anti-CTLA-4 or the combination. Results are the sum of two independent experiments with 7-8 mice/group.

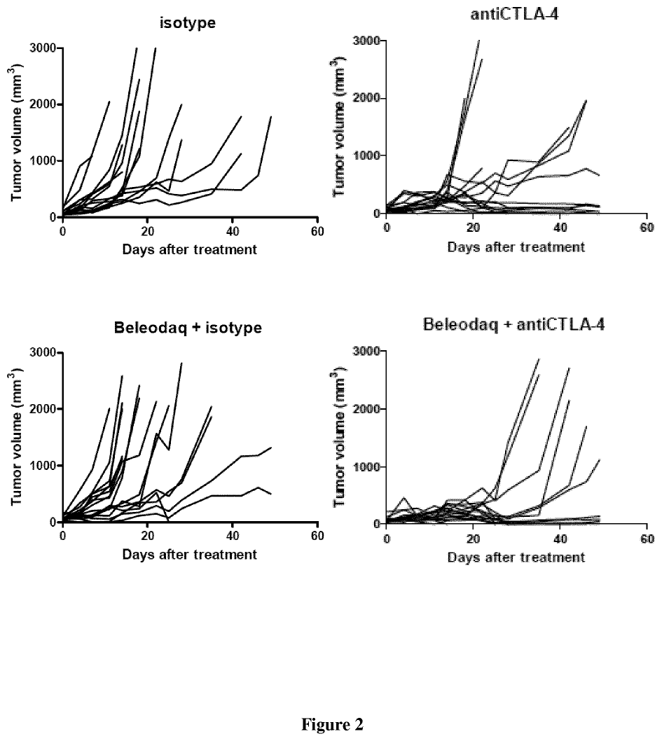

FIG. 3. provides the results for survival during treatment with (A) Belinostat (Beleodaq), anti-PD-1 or the combination and (B) with anti-CTLA-4 or the combination in an orthotopic breast cancer model. Data are plotted as the % of the group living on each day.

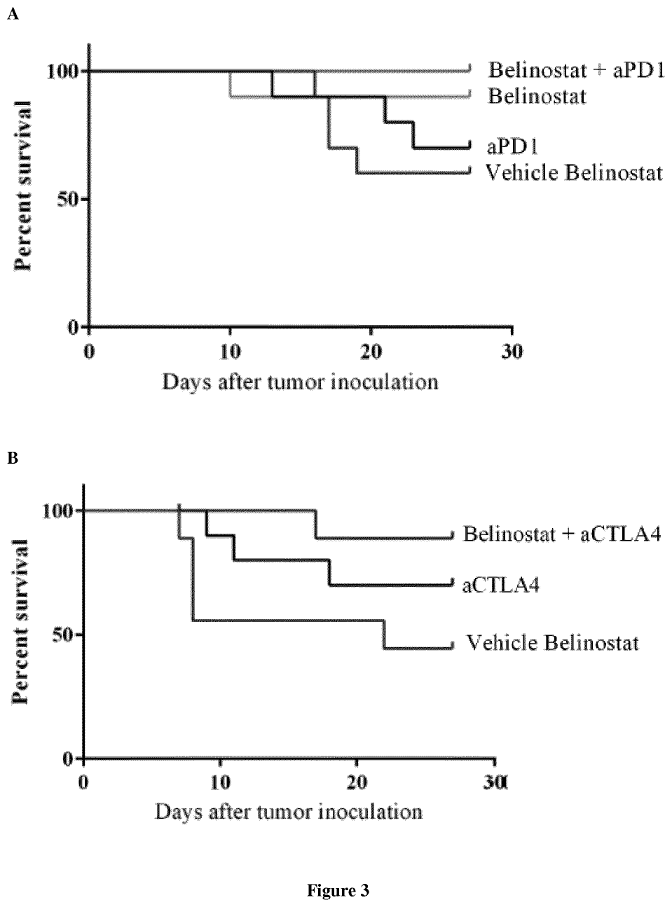

### DETAILED DESCRIPTION

In a hepatocellular carcinoma (HCC) mouse model, the inventors demonstrated that belinostat improves anti-tumor therapeutic response induced by the anti-CTLA-4 immune checkpoint inhibitor with a significant superior tumor growth inhibition compared to control groups. Treatment with the combination results in a complete cessation in tumor growth in all mice during the belinostat treatment period which continues for 1 week after the final dose. Mechanistic studies shows that the underlying immune response correlated with the observed therapeutic effect of the combination with enhancement of IFN-gamma production as antitumor T cell response and decrease in regulatory T cells in the spleens of treated animals.

These results provide a rational for using belinostat in combination with immune checkpoint inhibitors to reinforce therapeutic response. Currently only approximately 20% of patients respond to immune checkpoint inhibitors alone whereas combinations with belinostat leads to a significant in number of patients exhibiting significant treatment responses (increase in the number of responders). Belinostat is a booster of anti-tumor activity when combined with immunomodulatory agents, especially immune checkpoint inhibitors such as anti-CTLA-4 antibodies.

These first results have then been confirmed in other cancer mouse model (a breast cancer mouse model) since the inventors demonstrated that belinostat in combination with immune checkpoint inhibitors (both anti-PD-1 and anti-CTLA-4 antibodies) improves time survival of treated mice.

### Combinations of Immuno-oncology Agents and Anti-Cancer Agents

In certain embodiments, the present disclosure is directed to pharmaceutical combinations comprising one or more immuno-oncology agents, such as immunomodulatory compounds or compositions, in combination with one or more anti-cancer agents (i.e., compounds or compositions). Immunomodulatory compounds modulate the response of the immune system of the patient to be treated, typically activating or enhancing the response. While often providing therapeutic benefit by themselves, immunomodulatory compounds also exhibit a synergistic effect when administered in combination with belinostat as described herein.

### Immunomodulatory Compounds and Compositions

The term “immunomodulatory compound,” as used herein, refers to an agent that increases or enhances an immune response in the body (e.g., anti-tumor immune response). Exemplary immunomodulatory compounds of the present disclosure include antibodies, such as an anti-CTLA-4 antibody, anti-PD-1 antibody, an anti-PD-Ll antibody, and fragments thereof.

In certain embodiments, the present pharmaceutical combinations comprise an immunomodulatory compound that is an immune checkpoint inhibitor (ICI) or an immune checkpoint stimulator (ICS). In some embodiments, the ICI or ICS is an antibody.

The term “antibody,” as used herein, refers to an immunoglobulin or a fragment or a derivative thereof, and encompasses any polypeptide comprising an antigen-binding site, regardless of whether it is produced in vitro or in vivo. The term includes, but is not limited to, polyclonal, monoclonal, monospecific, polyspecific, nonspecific, humanized, single-chain, chimeric, synthetic, recombinant, hybrid, mutated, and grafted antibodies. The term “antibody” also includes antibody fragments such as Fab, F(ab′)2, Fv, scFv, Fd, dAb, and other antibody fragments that retain antigen-binding function, i.e., the ability to bind, for example, CTLA-4, PD-1, or PD-L1, specifically. Typically, such fragments comprise an antigen-binding domain.

In certain embodiments, the immunomodulatory compound is an ICI.

In such embodiments, the ICI may be an antibody, such as antibodies selected from anti-CTLA-4 antibodies, anti-PD-1 antibodies, and anti-PD-L1 antibodies. In other embodiments, the immunomodulatory compound is an ICI that is an antibody selected from anti-KIR antibodies (such as Lirilumab by Innate Pharma & BMS), anti-BTLA antibodies, anti-HVEM antibodies, anti-LAG3 antibodies (such as BMS-986016 by BMS), anti-TIM3 antibodies, and anti-NKG2A antibodies (such as monalizumab by Innate Pharma).

In certain embodiments, the immunomodulatory compound is an ICI that is an anti-CTLA-4 anti-body, such as ipilimumab (BMS/Yervoy®) or tremelimumab (Pfizer). In other embodiments, the ICI is an anti-PD-1 antibody, such as lambrolizumab-pembrolizumab (Merck/Keytruda®) or nivolumab (BMS/Opdivo®). In further embodiments, the ICI is an anti-PD-L1 antibody, such as avelumab (Merck), durvalumab (AstraZeneca), BMS-936559 (BMS), or atezolizumab MPDL3280A (Roche).

The term “anti-CTLA-4 antibody,” as used herein, refers to an antibody that selectively binds a CTLA-4 polypeptide. Exemplary anti-CTLA-4 antibodies are described for example in U.S. Pat. Nos. 6,682,736; 7,109,003; 7,123,281; 7,411,057; 7,824,679; 8,143,379; 7,807,797; and 8,491,895 which are incorporated by reference herein in their entirety. Tremelimumab is an exemplary anti-CTLA-4 antibody.

In certain embodiments, the immunomodulatory compound is an ICS.

In such embodiments, the ICS may be an antibody, such as an agonistic antibody directed against stimulatory checkpoint molecules. In some embodiments, the ICS is an antibody selected from anti-CD40 agonist antibodies (e.g. CP-870,893 (Pfizer and VLST), dacetuzumab (Seattle Genetics), Chi Lob 7/4 (University of Southampton) and lucatumumab (Novartis), anti-ICOS agonist antibodies, and anti-OX40 agonist antibodies (e.g. MEDI6469 (MedImmune).

In some embodiments, the present pharmaceutical combinations comprises one or more antibodies. For instance, in certain embodiments, the pharmaceutical combinations comprise the pharmaceutical combinations comprise two or more immune checkpoint inhibitors (ICI) (such as a CTLA-4 antibody and an anti-PD-1 antibody). In other embodiments, the pharmaceutical combinations comprise an immune checkpoint inhibitor (ICI) and an immune checkpoint stimulator (ICS) (such as an anti-PD-1 antibody and an anti-CD40 agonist antibody).

### Anti-Cancer Compounds and Compositions

In some embodiments, the anti-cancer compound or composition comprises belinostat or a pharmaceutically acceptable salt thereof or a pharmaceutical composition comprising thereof.

Belinostat, a histone deacetylase inhibitor, (also known as PXD-101) has the chemical name (2E)-N-hydroxy-3-[3-(phenylsulfamoyl)phenyl]prop-2-enamide and the following chemical formula:

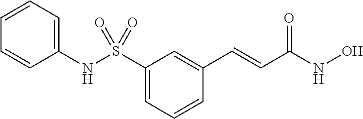 [SMILES: O=C(/C=C/c1cccc(S(=O)(=O)Nc2ccccc2)c1)NO]

Belinostat is currently commercially available for injection in the U.S. under the brand name Beleodaq® (Spectrum Pharmaceuticals, Henderson, Nev.).

The term “histone deacetylase inhibitor” or “HDACi,” as used herein, refers to a compound natural or manmade that inhibits histone deacetylase activity. There exist different classes of HDACi in function of their selectivity for their substrates. The term “histone deacetylase” or “HDAC,” as used herein, refers to an enzyme that removes acetyl groups from histones.

Belinostat and pharmaceuticals compositions comprising thereof useful in the present combinations are described in the international patent applications No WO 2002/30879 and WO 2006/120456, the contents of both of which are incorporated herein in their entirety. In certain embodiments, belinostat is formulated with arginine (such as L-arginine).

In some embodiments, the present pharmaceutical combinations comprise a therapeutically effective amount of belinostat and a therapeutically effective amount of an anti-CTLA-4 antibody. Preferably, the anti-CTLA-4 antibody is ipilimumab or tremelimumab. In a particular embodiment, the pharmaceutical combination comprises Beleodaq® and Yervoy®.

In other embodiments, the present pharmaceutical combinations comprise a therapeutically effective amount of belinostat and a therapeutically effective amount of an anti-PD-1 antibody. Preferably, the anti-PD-1 antibody is lambrolizumab-pembrolizumab or nivolumab.

In a particular embodiment, the pharmaceutical combination comprises Beleodaq® and Opdivo® or Beleodaq® and Keytruda®. In certain embodiments, the present pharmaceutical combinations comprise a therapeutically effective amount of belinostat, a therapeutically effective amount of an anti-CTLA-4 antibody and a therapeutically effective amount of anti-PD-1 antibody. In a particular embodiment, the pharmaceutical combination comprises Beleodaq®, Yervoy® and Opdivo®.

### Combinations of Anti-Cancer Agents

In certain embodiments, the present pharmaceutical combinations comprise two or more anti-cancer agents (i.e., compounds or compositions). For example, in some embodiments, the present combinations comprise nanoparticles of doxorubicin (such as doxorubicin-loaded poly(cyanoacrylate) nanoparticles) with belinostat or a pharmaceutically acceptable salt thereof.

In a particular embodiment, the present pharmaceutical combinations comprise doxorubicin-loaded poly(cyanoacrylate) nanoparticles, such as poly-isohexylcyanoacrylate (PIHCA) or poly-ethylbutylcyanocrylate (PEBCA), in combination with belinostat or a pharmaceutically acceptable salt thereof or a pharmaceutical composition comprising belinostat, such as a pharmaceutical composition comprising belinostat and arginine. In one such embodiment, the pharmaceutical combination comprises Livatag® (doxorubicin Transdrug™) and Beleodaq®.

Embodiments that employ two or more anti-cancer agents may also comprise an immuno-oncology agent, as described above.

### Kits and Compositions of the Present Pharmaceutical Combinations

In some embodiments, the components of the above-described pharmaceutical combinations are combined together into a single composition, such as a unit dosage form, such as a pill, tablet, capsule, lozenge, powder, solution, emulsion, suspension, cream, and the like. For example, in certain embodiments, the present pharmaceutical combinations comprise the one or more immuno-oncology agents and the one or more anti-cancer agents in a single composition or dosage form.

In other embodiments, the components of the above-described pharmaceutical combinations are provided as a kit. In one such embodiment, the kit contains the one or more immuno-oncology agents and the one or more anti-cancer agents in separate packaging or containers, optionally with instructions, such that the components are ready available for mixing or concomitant or sequential administration, as described below.

### Methods of Treating Cancer

In another aspect, the present disclosure provides methods for preventing or treating cancer by administering to a patient in need thereof a therapeutically effective amount of the pharmaceutical combinations described herein.

While certain of the immuno-oncology agents or anti-cancer agents described herein may be effective for preventing or treating various cancers in certain patients when administered individually, many patients do not respond to these monotherapies. By employing the present pharmaceutical combinations, the present methods are often able to overcome such treatment resistance, possibly through synergistic effects between the multiple agents/components employed in the present pharmaceutical combinations. The present methods are therefore useful for preventing or treating a variety of different cancers, as described below and shown in the examples.

In some embodiments, the cancer is selected from pancreatic cancer, liver cancer, breast cancer, prostate cancer, colon cancer, rectal cancer, colorectal cancer, lung cancer, head and neck cancer, melanoma, renal carcinoma, ovarian cancer, bone cancer, sarcoma, lymphoma, and leukemia. A particular example is hepatocellular carcinoma (HCC) or breast cancer.

In other embodiments, the present disclosure provides methods for treating HCC by administering to a patient in need thereof a therapeutically effective amount of the pharmaceutical combinations described herein. In a particular embodiment for treating HCC, the pharmaceutical combination comprises a therapeutically effective amount of belinostat and a therapeutically effective amount of an anti-CTLA-4 antibody. In one such embodiment, the method comprises administering a therapeutically effective combination of Beleodaq® and an anti-CTLA-4 antibody (such as Yervoy®).

Alternatively, the present disclosure provides methods for treating HCC by administering to a patient in need thereof a therapeutically effective amount of a histone deacetylase inhibitor (HDACi) and a therapeutically effective amount of an anti-CTLA-4 antibody. In one such embodiment, the method comprises administering other HDACi than belinostat. For instance, other examples of HDCAi suitable for use in the present pharmaceutical combinations include hydroxamic acids or hydroxamates (such as trichostatin A, vorinostat (Zolinza®), LAQ824, and panobinostat (Farydak®), cyclic tetrapeptides (such as trapoxin B), depsipeptides, benzamides (such as entinostat and mocetinostat), electrophilic ketones, and aliphatic acid compounds (such as phenylbutyrate and valproic acid). In such embodiments, the method comprises administering a therapeutically effective combination of Zolinza® and an anti-CTLA-4 antibody (such as Yervoy®) or a therapeutically effective combination of Farydak® and an anti-CTLA-4 antibody (such as Yervoy®).

The present disclosure also relates to a pharmaceutical combination comprising a therapeutically effective amount of HDACi and a therapeutically effective amount of an anti-CTLA-4 antibody accordingly. For instance, in certain embodiments, the pharmaceutical combination comprises a therapeutically effective amount of HDACi (such as vorinostat, panobinostat, entinostat, mocetinostat) and a therapeutically effective amount of an anti-CTLA-4 antibody. In one embodiment, the method comprises administering a therapeutically effective combination of Farydak® and an anti-CTLA-4 antibody (such as Yervoy®). In another embodiment, the method comprises administering a therapeutically effective combination of Zolinza® and an anti-CTLA-4 antibody (such as Yervoy®).

In other embodiments, the present disclosure provides methods for treating breast cancer by administering to a patient in need thereof a therapeutically effective amount of the pharmaceutical combinations described herein. In a particular embodiment for treating breast cancer, the pharmaceutical combination comprises a therapeutically effective amount of belinostat and a therapeutically effective amount of an anti-CTLA-4 antibody. In one such embodiment, the method comprises administering a therapeutically effective combination of Beleodaq® and an anti-CTLA-4 antibody (such as Yervoy®). In another particular embodiment for treating breast cancer, the pharmaceutical combination comprises a therapeutically effective amount of belinostat and a therapeutically effective amount of an anti-PD-1 antibody. In one such embodiment, the method comprises administering a therapeutically effective combination of Beleodaq® and an anti-PD-1 antibody (such Keytruda® or Opdivo®).

The invention relates to a method for the treatment of a cancer, to a pharmaceutical combination, or a kit as disclosed above, wherein the immunomodulatory compound (for instance an anti-CTLA-4 antibody) is used at lower dosage than the conventional dosage used in immunotherapy for the same indication and the same administration route when it is used alone (i.e., an amount equal to or preferably lower than the one used in conventional immunotherapy), also called herein a sub-therapeutic amount. More particularly, the amount can be for instance 90, 80, 70, 60, 50, 40, 30, 20 or 10% of the conventional therapeutic dosage (in particular for the same indication and the same administration route). The conventional therapeutic dosages are those acknowledged by the drug approvals agencies (e.g., FDA or EMEA). In that respect, the invention relates to a method for the treatment of a cancer, to a pharmaceutical combination or kit as disclosed above, wherein the amount of the immunomodulatory compound is used at a sub-therapeutic dosage and the amount of belinostat or a pharmaceutical salt thereof as disclosed herein is such that the combined therapeutic effect of the two active ingredients is additional or more preferably synergistic.

By the term “synergistic” therapeutic effect is meant that the obtained therapeutic effect of the combination is more than the addition of the therapeutic effect of each partner alone (i.e. more than the effect of belinostat or a pharmaceutical salt thereof as disclosed herein alone plus the effect of the immunomodulatory compound alone). By the term “additional” therapeutic effect is meant that the obtained therapeutic effect of the combination is the addition of the therapeutic effect of each partner alone (i.e. equals to the effect of belinostat or a pharmaceutical salt thereof as disclosed herein alone plus the effect of the immunomodulatory compound alone).

The invention also relates to a pharmaceutical combination wherein the amount or dosage of the immunomodulatory compound (e.g. an anti-CTLA-4 antibody) can be lowered in comparison with its amount or dosage when it is used alone. Indeed, the combination of belinostat or a pharmaceutical salt thereof and an immunomodulatory compound leads at least to an additive effect but rather to a clear synergistic effect of the two active ingredients. Then, with the pharmaceutical combination of the invention, it is possible to preserve the efficacy of the treatment, or even to improve it, while decreasing its adverse effects, in particular the adverse effects of the immunomodulatory compound. Alternatively, instead of lowering the amount or dosage of the immunomodulatory compound, the administration frequency of the immunomodulatory compound or its or treatment period can be reduced. According to an embodiment, the invention relates to a method for the treatment of a cancer, to a pharmaceutical combination preparation as disclosed above, wherein the amounts of belinostat or a pharmaceutical salt thereof as disclosed herein and the immunomodulatory compound in the pharmaceutical combination are such that the combined therapeutic effect of the two active components is additional or preferably synergistic.

In some embodiments, the present disclosure provides methods for preventing or reducing the recurrence of cancer relapse by administering to a patient in need thereof a therapeutically effective amount of the pharmaceutical combinations described herein.

In other embodiments, present disclosure provides methods for increasing survival time of a patient suffering from cancer by administering to the patient a therapeutically effective amount of the pharmaceutical combinations described herein. In such embodiments, the survival time can be measured as Progression-Free Survival (PFS) or Overall Survival (OS) or a combination thereof. In a particular embodiment, when the patient in need thereof is a patient suffering from HCC, the pharmaceutical combination comprises a therapeutically effective amount of belinostat and a therapeutically effective amount of an anti-CTLA-4 antibody. In one such embodiment, the method comprises administering a therapeutically effective combination of Beleodaq® and an anti-CTLA-4 antibody (such as Yervoy®). In a particular embodiment, when the patient in need thereof is a patient suffering from breast cancer, the pharmaceutical combination comprises a therapeutically effective amount of belinostat and a therapeutically effective amount of an anti-CTLA-4 antibody. In one such embodiment, the method comprises administering a therapeutically effective combination of Beleodaq® and an anti-CTLA-4 antibody (such as Yervoy®). In another particular embodiment for treating breast cancer, the pharmaceutical combination comprises a therapeutically effective amount of belinostat and a therapeutically effective amount of an anti-PD-1 antibody. In one such embodiment, the method comprises administering a therapeutically effective combination of Beleodaq® and an anti-PD-1 antibody (such Keytruda® or Opdivo®).

In other embodiments, present the disclosure provides for increasing anti-tumor activity in a patient in need thereof by administering to the patient a therapeutically effective amount of a pharmaceutical combination described herein. In a particular embodiment, when the patient in need thereof is a patient suffering from HCC, the pharmaceutical combination comprises a therapeutically effective amount of belinostat and a therapeutically effective amount of an anti-CTLA-4 antibody. In one such embodiment, the method comprises administering a therapeutically effective combination of Beleodaq® and an anti-CTLA-4 antibody (such as Yervoy®).

In other embodiments, present disclosure provides for increasing an anti-tumor immune response in a patient in need thereof by administering to the patient a therapeutically effective amount of a pharmaceutical combination described herein. In a particular embodiment, when the patient in need thereof is a patient suffering from HCC, the pharmaceutical combination comprises a therapeutically effective amount of belinostat and a therapeutically effective amount of an anti-CTLA-4 antibody. In one such embodiment, the method comprises administering a therapeutically effective combination of Beleodaq® and an anti-CTLA-4 antibody (such as Yervoy®).

The term “anti-tumor activity,” as used herein, refers to any biological activity that reduces or stabilizes the proliferation or reduces the survival of a tumor cell. In one embodiment, the anti-tumor activity is an anti-tumor immune response.

In some embodiments, the present disclosure relates to a pharmaceutical combination comprising a therapeutically effective amount of belinostat and a therapeutically effective amount of an anti-CTLA-4 antibody for use in a method for preventing or treating HCC in a patient in need thereof. The invention also relates to a pharmaceutical combination comprising a therapeutically effective amount of belinostat and a therapeutically effective amount of an anti-CTLA-4 antibody for use in a method for increasing an anti-tumor immune response in a patient in need thereof. In one embodiment, the patient in need thereof is a patient suffering from HCC. The invention further relates to a pharmaceutical combination comprising a therapeutically effective amount of belinostat and a therapeutically effective amount of an anti-CTLA-4 antibody for use in a method for increasing an anti-tumor immune response in a patient in need thereof. In one embodiment, the patient in need thereof is a patient suffering from HCC.

In some embodiments, the present disclosure relates to a pharmaceutical combination comprising a therapeutically effective amount of belinostat and a therapeutically effective amount of an anti-CTLA-4 antibody or of an anti-PD-1 antibody for use in a method for preventing or treating breast cancer in a patient in need thereof. The invention also relates to a pharmaceutical combination comprising a therapeutically effective amount of belinostat and a therapeutically effective amount of an anti-CTLA-4 antibody or of an anti-PD-1 antibody for use in a method for increasing survival time in a patient in need thereof.

### Regimen, Dosages and Administration Routes

The effective dosage of each of the combination partners/components employed in the pharmaceutical combinations of the invention may vary depending on the particular compound or pharmaceutical composition employed, the mode of administration, the condition being treated, the severity of the condition being treated. Thus, the dosage regimen of the pharmaceutical combination of the invention is selected in accordance with a variety of factors including the route of administration and the patient status. A physician, clinician or veterinarian of ordinary skill can readily determine and prescribe the effective amount of the single active ingredients required to prevent, counter or arrest the progress of the condition. Optimal precision in achieving concentration of the active ingredients within the range that yields efficacy without toxicity requires a regimen based on the kinetics of the active ingredients' availability to target sites.

As discussed above, the combined therapeutic effect of the two components employed in the pharmaceutical combinations of the invention is additional or more preferably synergistic.

Determining an additional or a synergistic interaction between two components, the optimum range for the effect and absolute dose ranges of each component for the effect may be definitively measured by administration of the components over different w/w ratio ranges and doses to patients in need of treatment. For humans, the complexity and cost of carrying out clinical studies on patients may render impractical the use of this form of testing as a primary model for synergy. However, the observation of synergy in one species can be predictive of the effect in other species and animal models exist to measure a synergistic effect and the results of such studies can also be used to predict effective dose and plasma concentration ratio ranges and the absolute doses and plasma concentrations required in other species by the application of pharmacokinetic/pharmacodynamic methods. Correlations between cancer models and effects seen in man suggest that observed synergy on animal models may be predictive of a synergy on man too.

The pharmacological activity of a combination of the invention may, for example, be demonstrated in a clinical study or more preferably in a test procedure. Suitable clinical studies are, for example, open label non-randomized, dose escalation studies in patients with advanced tumors. Such studies can prove the additive or synergism of the active ingredients of the combination of the invention. The beneficial effects on proliferative diseases can be determined directly through the results of these studies or by changes in the study design which are known as such to a person skilled in the art. Such studies are, in particular, suitable to compare the effects of a monotherapy using the active ingredients and a combination of the invention. Preferably, the combination partner (a) is administered with a fixed dose and the dose of the combination partner (b) is escalated until the maximum tolerated dosage is reached. Alternatively, the combination partner (b) is administered with a fixed dose and the dose of the combination partner (a) is escalated until the maximum tolerated dosage is reached.

As discussed above, in some embodiments the present pharmaceutical combinations are provided as a single composition, such as a solid or liquid composition, such as a solid or liquid unit dosage form. The components of such combinations (e.g., the immuno-oncology agent and the anti-cancer agent) are therefore simultaneously administered in the single composition.

In other embodiments, the components of the present pharmaceutical combinations are provided as a kit or separately, such as in separate compositions or dosage forms. In such embodiments, administration of the components (e.g., the immuno-oncology agent or the anti-cancer agent) may be via concurrent administration or sequential administration. “Concurrent administration,” as used herein, refers to the administration of the agents in separate unit dosage forms within a short period of time of one another, such as within about 0.5, 1, 2, 5, 10, 15, 30, or 60 minutes, or essentially administering the two drugs at the same time but in different dosage forms. “Sequential administration,” as used herein, refers to administration of one agent followed by administration of another agent with a longer intervening period of time, e.g., 1, 2, 3, 4, 6, or 12 hours, or the period of time between administrations may be extended, e.g., days, weeks, etc. In certain embodiments, two agents may be administered by one or more methods of simultaneous, concurrent, and sequential administration.

Regardless of whether simultaneous, concurrent, or sequential administration is employed, the compositions may be administered orally, sublingually, parenterally (e.g., intravenous, intraarterial, subcutaneous, and intramuscular injection), rectally, and nasally as appropriate for the particular composition.

Further aspects and advantages of the invention will be described in the following examples, which should be regarded as illustrative and not limiting.

### EXAMPLES

### Example 1: Hepatocellular Carcinoma (HCC) Mouse Model

### Materials and Methods

### Cell Injection

As tumor model, Hepa129 murine hepatocellular carcinoma cells implanted subcutaneously in C3H mice were used. Hepa129 cells were maintained in culture up to 3 passages before injection in mice. Cells were sub-cultured three days before the day of injection.

The day of cell injection, Hepa129 cells were harvested and centrifuged at 1800 rpm for 5 min, and resuspended in PBS. Cells were counted in 0.04% Trypan Blue for viability quantification. Cells were resuspended in the adequate volume of PBS in order to have 10⁶ cells in 100 μL. 100 of cells were subcutaneously injected into the right flank of mice.

### Treatment

When subcutaneous Hepa129 tumors reached a mean tumor diameter of about 5 mm (day 7), mice were allocated to treatment groups and identified individually by ear tags. Beleodaq® was administered IP daily at days 7 to 27 at 90 mg/kg. Antibodies Isotype control (2A3) and anti-CTLA-4 (9D9) (from BioXcell) were administered IP at days 7 and 14.

Treatment allocation were performed as follows for antitumor efficacy study:

| | No of | | | | |
|---|---|---|---|---|---|
| | animals | | | | |
| | per | | | | Treatment |
| Group | group | Treatment | Route | Dose | schedule |
| 1 | 8 | Isotype | IP | 50 μg/mouse | D 7, D 14 |
| 2 | 8 | Anti-CTLA-4 | IP | 50 μg/mouse | D 7, D 14 |
| 3 | 8 | Isotype + | IP + | 50 μg/mouse + | D 7, D 14 + |
| | | Beleodaq ® | IP | 90 mg/kg | D 7 to D 27 |
| 4 | 8 | Anti-CTLA-4 + | IP + | 50 μg/mouse + | D 7, D 14 + |
| | | Beleodaq ® | IP | 90 mg/kg | D 7 to D 27 |

Animals were monitored and sacrificed when tumor diameter reached 17 mm. Each experiment was carried out twice with n=8 animals per group.

Treatment allocation were performed as follows for immune characterization study:

| | No of | | | | |
|---|---|---|---|---|---|
| | animals | | | | |
| | per | | | | Treatment |
| Group | group | Treatment | Route | Dose | schedule |
| 1 | 5 | Isotype | IP | 50 μg/mouse | D 7, D 14 |
| 2 | 5 | Anti-CTLA-4 | IP | 50 μg/mouse | D 7, D 14 |
| 3 | 5 | Isotype + | IP + | 50 μg/mouse + | D 7, D 14 + |
| | | Beleodaq ® | IP | 90 mg/kg | D 7 to D 21 |
| 4 | 5 | Anti-CTLA-4 + | IP + | 50 μg/mouse + | D 7, D 14 + |
| | | Beleodaq ® | IP | 90 mg/kg | D 7 to D 21 |

A single experiment was carried out for each drug, using n=5 animals per group.

### Immune Characterization

Lymphoid organs (spleen, lymph nodes) were obtained from sacrificed animals, homogenized and cell suspensions were used to measure the following parameters:

Flow cytometry were used to determine:
% of CD4 and CD8 T cells (defined as CD3+ CD4+or CD3+CD8+ cells);
% of regulatory T cells (defined as CD4+, CD25+, FoxP3+ cells); and
% of myeloid-derived suppressor cells (defined as CD11b+ Ly6C+ for monocytic MDSC and CD11b+ Ly6G+ for granulocytic MDSC).

ELISPOT: measuring the number of IFN-gamma spot forming cells after co-culture of lymphocytes with irradiated Hepa129 tumor cells.

### Results

### Antitumor Efficacy

Animals were treated with the different combinations according to the protocols described above, including also control groups containing animals treated with an isotype control antibody or with monotherapies (control antibody plus the drug to be tested (Beleodaq®), therapeutic antibodies (anti-CTLA-4)).

Control animals treated with Isotype control antibody showed normal tumor growth and survival curves. Administration of Beleodaq® did not modify tumor growth rate and animal survival, with results equivalent to those observed in control mice. Administration of antibodies against CTLA-4 decreased tumor growth and prolonged animal survival. When combining Beleodaq® with therapeutic antibodies, Beleodaq® improved antitumor responses in combination with anti-CTLA-4. In this case, there was a delay in tumor growth observable until day 28 (approximately one week after the end of Beleodaq® treatment), when tumor growth increased, reaching levels equivalent to those observed in the group treated with anti-CTLA-4 monotherapy. Similarly, whereas first mice treated with anti-CTLA-4 had to be sacrificed at days 18-20, 100% of mice still survived until day 35. These results suggest that Beleodaq® improves the antitumor therapeutic response induced by anti-CTLA-4 antibodies.

### Characterization of Immune Mechanisms

Tumor-bearing mice were treated during two weeks and sacrificed to obtain their spleen and analyze immune-related parameters. For these experiments mice received two antibody administrations (days 0 and 7) and Beleodaq® (daily) during the two-week treatment period.

Functional and phenotypical analyses were carried out using splenocytes:

Functional assays: Spleen cells were stimulated with irradiated tumor cells to analyze antitumor T cell responses measured as IFN-gamma production using a 20-h ELISPOT assay. Although no significant differences were observed in Isotype-treated group after combination with Beleodaq®, mice receiving anti-CTLA-4+ Beleodaq® had significantly higher responses than those treated with anti-CTLA-4 monotherapy.

Phenotypical analyses: By employing commonly used phenotypical markers, the proportion of different splenic cell subsets was determined. Initial characterization involved T cells: percentages of total CD4, CD8 as well as T regulatory cells were analyzed. Although no differences were observed between groups in total CD4 or CD8 T cells, a significant decrease in Tregs was found in mice treated with anti-CTLA-4+ Beleodaq®, in comparison with those treated only with anti CTLA-4.

Analyses of PD-1 and ICOS on T cells showed a low expression of these markers in CD8 T cells, with no significant differences between groups. Regarding CD4 T cells, a higher percentage of PD-1+ cells and ICOS+ cells were observed in the anti-CTLA-4 monotherapy group, compared with anti-CTLA-4+ Beleodaq® combination. Differences corresponded to effector CD4 T cells, since no differences in PD-1 expression were observed in T regulatory cells.

Finally, percentage of myeloid cell subsets, including myeloid-derived suppressor cells were quantified in the spleen. Although a general trend showing an increase in CD11b cells was observed in all groups treated with Beleodaq®, these values only reached statistical significance when comparing anti-CTLA-4 treated mice (with or without Beleodaq®) in the case of monocytic MDSC, but not in total CD11b+ or in granulocytic MDSC.

### Conclusion

When combined with anti-CTLA-4 antibodies, Beleodaq® decreased tumor growth rate and prolonged animal survival during the Beleodaq® treatment period over monotherapy. Combination of Beleodaq® with anti-CTLA-4 antibodies was also associated with an increased proportion of splenic tumor-specific T cells producing IFN-gamma and decreased proportion of T regulatory cells and PD-1+ effector T cells. Therefore, the Beleodaq® + anti-CTLA-4 combination shows a therapeutic anti-tumor effect in the Hepa129 HCC s.c. model.

### Example 2: Breast Cancer Mouse Model (4T1 cells)

### Materials and Methods

TABLE 1
Treatment/test articles. Dose and treatment details.

| Treat- | | Formula- | | Dose and | Administra- |
|---|---|---|---|---|---|
| ment | Test | tion | | administration | tion |
| name | article | vehicle | Route | pattern | volume |
| Ve- | n.a. | 32 mg/ml | i.p. | n.a., daily from | 5 |
| hicle¹ | | L-arginine | | day 8 on | ml/kg |
| | | in NaCl | | | |
| Belino- | Belino- | 32 mg/ml | i.p. | 80 mg/kg i.p. | 5 |
| stat | stat | L-arginine | | daily from day | ml/kg |
| | | in NaCl | | 8 on | |
| aPD1 | aPD1 | PBS | i.p. | 200 μg/mouse | 100 |
| | | | | i.p. on days 8, | μl/mouse |
| | | | | 12, 16 and 20 | |
| aCTLA4 | aCTLA4 | PBS | i.p. | 200 μg/mouse | 100 |
| | | | | i.p. on days | μl/mouse |
| | | | | 11, 16, 20 | |

Belinostat: Onxeo PXD02-001
aPD1: anti mouse PD-1 (Bio X Cell/Clone RMP1-14/Cat. no: BE00146).
aCTLA4: mouse anti mouse CTLA-4 (Bio X Cell/Clone 9D9/Cat. no: BE0164).

The Belinostat treatment was formulated every one or two day.

The aPD1 treatment solutions were prepared on the days of the treatment (days 8, 12, 16 and 20).

The aCTLA4 treatment solutions were prepared on days 7, 11, 16 and 20 and stored at 4° C.

### Model

4T1 breast cancer cells (5*10⁵ cells per Balb/C mouse) were in occulated in the mammary fat gland of Balb/C females. The 4T1 (ATCC CRL-2539) cells were bought from ATCC and cultured in RPMI-1640 Medium containing 10% fetal bovine serum. Cells were passaged 5-10 times before in inoculation. Cells were detached with trypsin, washed twice in PBS, centrifuged and resuspended in PBS. Cell viability was checked by a staining with tryptophan blue and cells were used when viability was higher than 95%. Cells were counted manually and adjusted to 5*10⁶ cells per ml. Finally 100 μl were injected.

### Results

The 4T1 breast cancer model is metastatic. In the present study, both the application of belinostat and antibody therapy reduced mortality and apparent external tumor size. These data show that the combination of belinostat with anti-PD-1 or anti-CTLA-4 antibodies is associated with a longer survival time.

While various specific embodiments have been illustrated and described, it will be appreciated that various changes can be made without departing from the spirit and scope of the claimed invention(s).

1. A pharmaceutical combination comprising at least one immunomodulatory compound and belinostat or a pharmaceutically acceptable salt thereof.

2. The pharmaceutical combination according to claim 1, wherein the immunomodulatory compound is an immune checkpoint inhibitor (ICI) selected from the group consisting of anti-CTLA-4 antibodies, anti-PD-1 antibodies, and anti-PD-L1 antibodies.

3. The pharmaceutical combination according to claim 2, wherein the ICI is an anti-CTLA4 antibody.

4. The pharmaceutical combination according to claim 3, wherein the ICI is an anti-CTLA4 antibody selected from ipilimumab and tremelimumab.

5. The pharmaceutical combination according to claim 2, wherein the ICI is an anti-PD1 antibody.

6. The pharmaceutical combination according to claim 5, wherein the ICI is an anti-PD1 antibody selected from lambrolizumab-pembrolizumab and nivolumab.

7. The pharmaceutical combination according to claim 1, wherein belinostat is formulated with arginine.

8. A pharmaceutical combination according to claim 1 for use in a method for treating cancer in a patient in need thereof.

9. The pharmaceutical combination for use according to claim 8, wherein the cancer is hepatocellular carcinoma (HCC).

10. The pharmaceutical combination for use according to claim 8, wherein the cancer is breast cancer.

11. A method for treating cancer in a patient in need thereof, comprising administering to the patient a therapeutically effective amount of the pharmaceutical combination according to claim 1.

12. The method according to claim 11, wherein the cancer is hepatocellular carcinoma (HCC).

13. The method according to claim 11, wherein the cancer is breast cancer.

14. A method for treating HCC in a patient in need thereof, comprising administering to said patient a therapeutically effective amount of HDACi inhibitor and a therapeutically effective amount of an anti-CTLA-4 antibody.

15. The method according to claim 14, wherein the HDACi inhibitor is selected from the group consisting of belinostat, trichostatin A, vorinostat, LAQ824, panobinostat, cyclic tetrapeptides, depsipeptides, entinostat, mocetinostat, phenylbutyrate and valproic acid.

16. The method according to claim 14, wherein the HDACi inhibitor is belinostat.

17. A method for increasing anti-tumor activity and/or anti-tumor immune response in a patient suffering from cancer comprising administering to the patient a therapeutically effective amount of belinostat and a therapeutically effective amount of an immunomodulatory compound.

18. The method according to claim 17, wherein the immunomodulatory compound is an anti-CTLA-4 antibody.

19. The method according to claim 17, wherein the patient is suffering from HCC.
